(19) United States
(12) Patent Application Publication
(10) Pub. No.: US 2025/0173508 A1
(43) Pub. Date: May 29, 2025

(54) METHODS AND SYSTEM FOR PROVIDING A DATA ELEMENT FROM A CORPUS OF DATA

(71) Applicant: Siemens Healthineers AG, Forchheim (DE)

(72) Inventors: Gerhard KOHL, Neunkirchen am Brand (DE); Gerardo HERMOSILLO VALADEZ, West Chester, PA (US)

(73) Assignee: Siemens Healthineers AG, Forchheim (DE)

(21) Appl. No.: 18/956,504

(22) Filed: Nov. 22, 2024

(30) Foreign Application Priority Data
Nov. 24, 2023 (DE) 10 2023 211 714.2

(51) Int. Cl. G06F 40/284; G16H 10/60

(52) U.S. Cl. CPC G06F 40/284; G16H 10/60

(57) ABSTRACT

A method and corresponding systems for providing a mapping of an unstructured corpus of data onto a predetermined data structure are provided. The method comprises obtaining a prompt for providing the data element; accessing a corpus of data; providing a machine-learned function configured to identify data elements in corpora of data based on prompts; applying the machine-learned function to the corpus of data to identify at least one data element in the corpus of data corresponding to the prompt; determining a confidence measure for the identified at least one data element using a verification function, the verification function being independent from the machine-learned function; and providing the identified at least one data element as the data element based on the confidence measure.

### CROSS-REFERENCE TO RELATED APPLICATION(S)

The present application claims priority under 35 U.S.C. § 119 to German Patent Application No. 10 2023 211 714.2, filed Nov. 24, 2023, the entire contents of which is incorporated herein by reference.

### FIELD

Embodiments of the invention concern systems and methods for retrieving a data element from a corpus of data. In particular, embodiments of the invention concern the identification of data elements in a large, in particular, unstructured, corpus of data based on a prompt. Further, embodiments of the invention concern the mapping of data elements of a corpus of data onto a data structure. Further, embodiments of the invention concern workflows for verifying said data elements. Specifically, according to embodiments of the invention, the corpora of data and/or data elements concern healthcare data such as patient data.

Independent of the grammatical term usage, individuals with male, female or other gender identities are included within the term.

### RELATED ART

Machine-learned functions like large language models offer a huge variety of different applications in healthcare informatics. For instance, they can be used to provide an intuitive interface for a user to interact with the data available in a healthcare information system. Specifically, rather than searching for data elements like individual values or findings in the available studies or documents by her- or himself, a user might input high-level queries into a machine-learned function which then interacts with the various databases connected to the system to provide an answer to the query. Thereby, it is desirable that the answer is equally “high-level” as the query. That is, in the optimal case, an answer should be provided in a form of an actionable result which the user can readily use. For instance, the user might ask questions like “are there indications in the EMR that patient A suffers from disease B”. As an answer, the user would expect a list of indications.

Another use case of, in particular, large language models is mapping unstructured data onto a given data structure or bridging between different data formats. Thereby, the language understanding capabilities enable LLMs to identify overarching meanings and categories in data elements and assign these to data types of a data structure on that basis. In other words, large language models are capable to abstract. For instance, this makes it possible to map information comprised in an EMR of a patient to data types or categories as, e.g., comprised in medical ontologies (e.g., SNOMED) or data standards (e.g., FHIR).

### SUMMARY

One fundamental issue with machine-learned functions and, in particular, large language models is that these systems tend to hallucinate or pretend to have found information which is not there. Especially in the medical environment this considerably hampers the usefulness as flaws in the data processing may have severe consequences in the downstream healthcare processes including diagnosis and treatment of patients.

Embodiments of the invention provide systems and methods which allow for a more secure identification and retrieval of data elements from a corpus of data. For example, embodiments of the invention reduce an improper or wrong identification of data elements.

These and other objects are, in particular, solved by methods, systems, computer program products and computer-readable storage media according to the appended claims.

### BRIEF DESCRIPTION OF THE DRAWINGS

Characteristics, features and advantages of the above-described invention, as well as the manner they are achieved, become clearer and more understandable in the light of the following description of embodiments, which will be described in detail with respect to the figures. This following description does not limit the invention on the contained embodiments. Same components, parts or steps can be labeled with the same reference signs in different figures. In general, the figures are not drawn to scale. In the following:

FIG. 1 schematically depicts a system for providing a data element according to an embodiment,

FIG. 2 schematically depicts a system for providing a data element according to a further embodiment,

FIG. 3 schematically depicts a method for providing a data element according to an embodiment,

FIG. 4 schematically depicts data flows in a method for providing a data element according to an embodiment,

FIG. 5 schematically depicts a method for providing a data element according to an embodiment,

FIG. 6 schematically depicts data flows in a method for providing a data element according to an embodiment,

FIG. 7 schematically depicts a method for providing a data element according to an embodiment,

FIG. 8 schematically depicts optional methods steps in a method for providing a data element according to an embodiment,

FIG. 9 schematically depicts data flows in a method for providing a data element according to an embodiment,

FIG. 10 schematically depicts a method for providing a data element according to an embodiment,

FIG. 11 schematically depicts data flows in a method for providing a data element according to an embodiment, and

FIG. 12 schematically depicts a text generation function according to an embodiment.

### DETAILED DESCRIPTION

In the following, A technical solution according to example embodiments is described with respect to the claimed systems as well as with respect to the claimed methods. Features, advantages, or alternative embodiments described herein can likewise be assigned to other claimed objects and vice versa. In other words, claims addressing the inventive method can be improved by features described or claimed with respect to the systems. In this case, e.g., functional features of the methods are embodied by objective units or elements of the systems.

Features and alternate forms of embodiments of data structures and/or functions for methods and systems for providing can be transferred to analogous data structures and/or functions for methods and systems for providing trained functions. Analogous data structures can, in particular, be identified by using the prefix “training”. Furthermore, the trained functions used in methods and system for providing a text summary can, in particular, have been adjusted and/or trained and/or provided by methods and systems for adjustment of trained functions.

According to an aspect, a computer-implemented method for providing a data element is provided. The method comprises a plurality of steps. One step is directed to obtain a prompt for providing the data element. Another step is directed to access a corpus of data. Another step is directed to provide a machine-learned function configured to identify data elements in corpuses of data based on prompts. Another step is directed to apply the machine-learned function to the corpus of data so as to identify at least one data element in the corpus of data corresponding to the prompt. Another step is directed to determine a confidence measure for the identified at least one data element using a verification function, the verification function being independent from the machine-learned function. Another step is directed to provide the identified at least one data element as the data element based on the confidence measure.

The corpus of data may be stored in one or more databases. The corpus of data may be a subset of data stored in the one or more databases. Further the corpus of data may be an (entirety) of data stored in the one or more databases. The corpus of data may comprise a plurality of different data items (or sources) such as documents, files, e.g., log files, sets, tables, arrays, and so forth. Each data item comprises one or more individual data elements.

According to some examples, the corpus of data comprises personal information of a (single) person such as a patient, customer, employee, or the like.

According to some examples, accessing the corpus of data may comprise querying one or more databases for the corpus of data. For instance, querying may be based on a data identifier identifying the corpus of data in the one or more databases. According to some examples, the data identifier may be an accession number, a case number, a person or patient identifier and the like.

A data element may relate to a piece of data or piece of information comprised in the corpus of data. A data element may comprise one or more numbers, logical relations, words, and any combination thereof. A data element may correspond to a certain data type or data category. A data element may be seen as a realization of a certain data type.

A data element may have a verbatim equivalent in the corpus of data, e.g., in the form of a value or data points. Further, a data element may be derived from one or more data points. In particular, a data element may be more high-level than the data points in the corpus of data. For instance, a data element may indicate if a patient suffers from a certain disease, wherein, optionally, this assertion is based on a plurality of measurement values comprised in the corpus of data.

A prompt may be a request or instruction for the machine-learned function to retrieve a data element from the corpus of data. In particular, the prompt may be an instruction to retrieve, from the corpus of data, data elements corresponding to one or more data types.

According to other examples, the prompt may be a high-level prompt, e.g., comprising a natural language query directed to certain facts based on the corpus of data. Such high-level prompts may be “translated”, e.g., by the or another machine-learned function, into prompts directed to concrete data types.

The prompt may generally be in a format which is machine readable and understandable by the machine-learned function.

In general, a machine-learned function mimics cognitive functions that humans associate with other human minds. In particular, by training based on training data, the machine-learned function is able to adapt to new circumstances and to detect and extrapolate patterns. Other terms for machine-learned function, may be trained function, trained machine learning model, trained mapping specification, mapping specification with trained parameters, function with trained parameters, algorithm based on artificial intelligence, or machine learned algorithm.

In general, parameters of a machine-learned function can be adapted via training. In particular, supervised training, semi-supervised training, unsupervised training, reinforcement learning and/or active learning can be used. Furthermore, representation learning can be used. In particular, the parameters of the machine-learned function can be adapted iteratively by several steps of training. In the presented case, codes of the medical ontology could be attributed to sample queries by an expert. This annotated data can then be used as a ground truth to train the machine-learning function.

In particular, a machine-learned function can comprise a neural network, a support vector machine, a decision tree and/or a Bayesian network, and/or the trained function can be based on k-means clustering, Q-learning, genetic algorithms and/or association rules. In particular, a neural network can be a deep neural network, a convolutional neural network or a convolutional deep neural network. Furthermore, a neural network can be an adversarial network, a deep adversarial network and/or a generative adversarial network.

In particular, the machine-learned function is configured to search a corpus of data for data elements according to a prompt and/or data elements of a particular data type.

According to some examples, the machine-learned function may be a computer program product comprising machine-executable instructions, e.g., by a computing unit executing the method. According to some examples, providing the machine-learned function may comprise holding the machine-learned function available in a suitable storage accessible by a computing unit executing the method.

The verification function may be a computer program product comprising machine-executable instructions, e.g., for a computing unit executing the method. The verification function is configured to independently verify the processing result of the machine-learned function, that is, data elements or mappings of data elements provided by the machine-learned function. To this end, the verification function may be configured to take-in the processing results of the machine-learned function and the input based on which the machine-learned function provided the processing results. According to some examples, this may include the prompt or data type sought after and/or the corpus of data or any excerpt of the corpus of data.

The verification function may itself be a machine-learned function. As an alternative, the verification function may be rule-based.

The verification function being different from the machine-learned function may mean that both functions are independent from one another. This may mean that both functions employ a different way of and/or use different means for arriving at the respective processing result. This may mean that both functions are based on a different working principle or a different implementation or a different architecture. For instance, the machine-learned function may be based on a transformer architecture as herein described, while the verification function may rely on a neural network architecture without transformers.

According to other examples, the machine-learned function and the verification function may have the same basic structure/architecture/configurations but have been trained differently, e.g., based on different training data. In other words, the two functions may differ in the parameters adjusted in the training processes.

The confidence measure (or certainty measure, or confidence level, or certainty score, or trust level) may be conceived as an indication of how well the data element identified by the machine-learned function matches the prompt or the data type. In other words, the confidence measure may comprise a quality indicator for the processing result of the machine-learned function. According to some examples the confidence measure may be a “yes-or-no”-statement regarding the question whether or not the data element is (really) comprised in the underlying data.

A confidence measure, according to some examples, may comprise a numerical value or a collection of numerical values (e.g., a vector) that indicate, according to a model or algorithm, the degree of certainty or uncertainty of the processing result of the machine-learned function. According to some examples, obtaining the certainty measures may comprise assigning a confidence measure to each data element identified.

By providing an independent verification function, the data retrieval of the machine-learned function can be monitored. In other words, an independent and automated reviewing instance is provided which renders the identification and retrieval of data elements from a corpus of data more reliable. With that, end users do not need to interfere with the process as frequently. In return, the data retrieval becomes more efficient and more widely applicable also for more sensible questions as encountered in healthcare.

According to some examples, the step of providing may comprise providing the data element to a user via a user interface. Further, the step of providing may comprise providing the data element for further processing (e.g., by a further data processing function).

According to some examples, the step of providing may comprise providing the confidence measure together with the data element. With that, the confidence measure may be documented, bought to the attention of or user, and/or used for subsequent automated processing.

According to an aspect, the step of providing comprises accepting or rejecting the identified at least one data element based on the confidence measure.

According to an aspect, the step of providing comprises comparing the confidence measure to a predetermined criterion and only provide the identified data element if the confidence measure fulfills the predetermined criterion. According to some examples, the predetermined criterion may be a threshold. With that, only such data elements which meet a certain quality criterion may be provided (e.g., to a user or for further processing).

According to some examples, the step of accessing the corpus of data comprises accessing the corpus of data from one or more databases, the one or more databases being located within the premises of an organization (in particular: a healthcare organization), the machine-learned model is hosted within the premises of the organization, and the verification function is hosted outside of the premises of the organization.

In other words, the machine-learned function may be self-hosted by the organization while the verification function is externally hosted outside of the organization, e.g., in a cloud or a webserver. The verification function may be provided in the form of a software as service or as a web-service. Accordingly, the machine-learned function and the verification function may be different in their hosting location. Hosting may mean providing the infrastructure (computing capabilities, storages) for applying/running the respective function. Hosting a function within the premises of an organization means that the infrastructure is provided within the organization. Hosting a function outside of the premises of an organization means that the infrastructure is outside of the premises.

Using a self-hosted machine-learned function for the initial identification of data elements has the advantage that the machine-learned function may be specifically adapted according to the needs of the organization. This may include adapting the machine-learned function to be able to identify data elements according to a specific data structure used in the organization. In turn, using an external verification function has the advantage of a reviewing instance which is unbiased by any local adaptation. At the same time, using a self-hosted machine-learned function may prevent that sensible personal data leaves the organization.

According to an aspect, the corpus of data comprises unstructured natural language text, and the machine-learned function is configured to identify data elements in natural language text.

In other words, the machine-learned function may comprise natural language understanding capabilities. Using a machine-learned function capable of identifying data elements in natural language text has the advantage that data can be extracted from unstructured text comprised in the corpus of data. Unstructured text is difficult to process by conventional rule-based functions as such data does not include defined data fields or pointers for the data elements.

According to some examples, the machine-learned function comprises a transformer network and/or a large language model.

A transformer network is a neural network architecture generally comprising an encoder, a decoder or both an encoder and decoder. In some instances, the encoders and/or decoders are composed of several corresponding encoding layers and decoding layers, respectively. Within each encoding and decoding layer is an attention mechanism. The attention mechanism, sometimes called self-attention, relates data items (such as words) within a series of data items to other data items within the series. The self-attention mechanism for instance allows the model to examine a group of words within a sentence and determine the relative importance other groups of words within that sentence have to the word being examined.

The encoder, in particular, may be configured to transform the input (text) into a numerical representation. The numerical representation may comprise a vector per input token (e.g., per word). The encoder may be configured to implement an attention mechanism so that each vector of a token is affected by the other tokens in the input. In particular, the encoder may be configured such that the representations resolve the desired output of the transformer network.

The decoder, in particular, may be configured to transform an input into a sequence of output tokens. In particular, the decoder may be configured to implement a masked self-attention mechanism so that each vector of a token is affected only by the other tokens to one side of a sequence. Further, the decoder may be auto-regressive meaning in that intermediate results (such as a previously predicted sequence of tokens) are fed back.

According to some examples, the output of the encoder is input into the decoder.

Further, the transformer network may comprise a classification module or unit configured to map the output of the encoder or decoder to a set of learned outputs such as the text summary.

Training of a transformer model according to some examples may happen in two stages, a pretraining and a fine-tuning stage. The fine-tuning stage may happen locally within the premises of the organization managing the corpus of data. In the pretraining stage, a transformer model may be trained on a large corpus of data to learn the underlying semantics of the problem. Such pre-trained transformer models are available for different languages. For certain applications described herein, the fine-tuning may comprise further training the transformer network with medical texts with expert annotated meanings and/or medical ontologies such as RADLEX and/or SNOMED. With the latter, in particular, the transformer model according to some examples may learn typical relations and synonyms of medical expressions.

For a review on transformer networks, reference is made to Vaswani et al., “Attention Is All You Need”, in arXiv: 1706.03762, Jun. 12, 2017, the contents of which are herein included by reference in their entirety.

An advantage of transformer networks is that, due to the attention mechanism, transformer networks can efficiently deal with long-range dependencies in input data. Further, encoders used in transformer networks are capable of processing data in parallel which saves computing resources in inference. Moreover, decoders of transformer networks, due the auto-regression, are able to iteratively generate a sequence of output tokens with great confidence.

A large language model (LLM) is a language model characterized by its large size. In particular, the large language model may be based on a transformer architecture. According to some examples, the large language model may comprise or may be based on models available at the date of filing such as GPT models (e.g., GPT-3.5 and GPT-4), PALM, LLaMa, LLaMa 2, Falcon, Whisper, and the like.

According to an aspect, the step of obtaining the prompt further comprises, obtaining a predetermined data structure with a plurality of data types, and defining the prompt as a prompt directed to identify data elements corresponding to one or more of the plurality of data types, wherein the confidence measure is indicative of how well the identified at least on data element corresponds to the corresponding data type.

A data structure may be conceived as a data organization, management, or/and storage format. Specifically, a data structure is a collection of data types, the relationships among them, and/or the functions or operations that can be applied to the data types. A data structure may be comprise or define a plurality of data fields for a plurality of data types. The data fields of a data structure may be populated by data elements of a certain realization or use case, i.e., a corpus of data. Assigning data elements of a corpus of data to data types of a data structure may also be referred to as “mapping” the corpus of data to the data structure.

According to some examples, the data structure may be specific for a certain organization. For instance, a medical record of the patient may be formatted according to a data structure. Then, the data fields/data types of suchlike medical record may be populated by data elements pertaining to the patient.

A data type may relate to a category of a data element. Taking personal data as an example, a data type may relate to the name, age, or any other demographic information of the person.

According to some examples, the data structure is based on or implements a predetermined standard, for instance a data communication standard or a standard data format.

Standard may mean that it is at least used within an organization as a standard. According to other examples, suchlike standards may also be used cross-organizational.

In other words, the machine-learned function is induced to map the corpus of data to a defined data structure. That way, the data structure may work as a scaffold for identifying data elements. This reduces the degrees of freedom of the machine-learned function and can improve the quality of the data retrieval. Further, it becomes possible to specifically train the machine-learned function to map data to the data structure which also improves the quality. As another advantage it becomes possible to use the processing for the task of formatting unstructured data according to a data structure for further processing. That way, it becomes possible to systematically access knowledge and systematically generate results which are directly actionable.

According to an aspect, the data structure is based on an ontology of data types and/or comprises a tree structure in which tree structure data types may be linked with edges.

An ontology may be a formally ordered representation of a set of data fields or data types and the relationships between them. In particular, the data types of a medical ontology may relate to concepts, data and/or other information. An ontology can be used in particular to exchange information in digitized and formal form between application programs and services. In particular, an ontology represents a network of concepts, data and/or information with logical relations. An ontology can, in particular, contain inference and integrity rules, i.e., rules for conclusions and for ensuring their validity. Further according to some examples, an ontology may comprise a hierarchy of data types where lower-level elements are linked to higher-level data types. In particular, an ontology may comprise a number of hierarchical levels. In an ontology, a data type may be characterized by its content as well as by its arrangement and relation to other data types in the ontology.

An ontology can be represented, in particular, in the form of a tree structure or mathematical graph comprising nodes and edges, in particular in the form of a directed graph. In this case, the nodes and/or the edges can, in particular, have further data types.

A data type may relate to a node (concept, data item and/or information) of the medical ontology. In particular, each data type may be configured to unambiguously identify a node (concept, data item or information) of the ontology. In particular, each data type may be configured to unambiguously identify a node (or edge) of the medical ontology.

The ontology may, in particular, be a medical ontology that relates to medical and/or (human) biological issues. A medical ontology is, in particular, independent of the specific patient, in other words, a medical ontology represents and structures existing abstract technical or domain knowledge. Such a medical ontology can be the result of scientific research, in particular with regard to the causal relationships and structure of medical information. In particular, the medical ontology is independent of the medical record database and the entries therein comprised.

According to some examples, the medical ontology is predefined. In particular, the medical ontology may be externally provided. According to some examples, the medical ontology may be public.

According to some examples, the step of identifying the at least one data element in the corpus of data may comprise identifying a notion relating to a data type of the data structure in the corpus of data and identifying the data element underlying the notion as corresponding to the data type.

One advantage of using ontologies is that by using existing and even public knowledge, a reliable and easy to implement way to query data systems for information can be provided. Further, by mapping to an ontology, an output can be provided which can readily be used for further processing such as answering user queries. This is because an ontology not only comprises data types such as an array of values but also relations between data types. Further, since an ontology is typically written for human users, it comprises high-level designations for the comprised data types (e.g., in the form of word descriptors). This simplifies the data element identification problem as the machine-learned function may search for these designations or synonyms in the corpus of data. This is even more so if the data structure comprises a tree structure since, in addition, relations between data types may be leveraged. In other words, using ontologies or tree structures as “targets” for the data element identification may give the machine-learned function additional clues where to look for the data elements in the corpus of data. This improves the quality and efficiency of the method.

According to an aspect, the machine-learned function is configured to provide a source in the corpus of data from which source the identified at least one data element was obtained, the verification function is further configured to provide confidence measures for identified data elements based on corresponding sources, and the step of determining the confidence measure comprises transmitting the source to the verification function.

A source may be conceived as an excerpt from the corpus of data. As such, a source may be a subset of the corpus of data. For instance, the source may be files, documents, or other portions of the corpus of data. The source may comprise the data element and additional information or data. The source may be seen as comprising (data) context for the data element. For instance, if the data element comprises one or more individual words or values, the source may comprise a text document or a text passage or snippet of the text document comprising the individual words or values.

By providing the source and feeding the source into the verification function, the verification function is provided with additional information about the basis for the “decision” of the machine-learned function. In other words, the source may be seen as “evidence” for the processing result of the machine-learned function. By relying on this evidence, the verification function does not have to search the entire corpus of data itself but only needs to selectively analyze relevant parts of it. This simplifies the processing and increases the efficiency of the method.

According to some aspects, the source is transmitted to the verification function (together with the prompt and the identified data item). According to some examples, the verification function does not have access to the corpus of data. Thus, in terms of underlying data, only the source is provided to the verification function. In other words, in the step of determining the confidence measure, the source is input into the verification function but not the (full) corpus of data.

With that, data traffic can be minimized and the computational effort when applying the verification function can be reduced. At the same time, this may increase the data security if the verification function is hosted outside of the premises of an organization as only a fraction of the full corpus of data needs to be transmitted to the outside of the organization.

According to some examples, the corpus of data comprises personal data and the method further comprises anonymizing and/or de-identifying the source, in particular, prior to transmitting the source to the verification function. Thereby, the verification function may be further configured to provide confidence measures for identified data elements based on corresponding anonymized and/or de-identified sources, and the step of determining the confidence measure comprises transmitting the anonymized and/or de-identified source to the verification function.

With that, it can be further ensured that no sensible data is transmitted to the verification function which may be non-proprietary of an organization administrating the corpus of data and/or be hosted outside of the organization.

According to an aspect, the machine-learned function is configured to provide a source in the corpus of data from which source the identified at least one data element was obtained, the verification function is configured to derive, based on identified data elements and corresponding sources, detailed source information indicating one or more portions within corresponding sources from which data elements have been obtained, the step of determining the confidence measure comprises deriving a detailed source information for the identified at least one data element, and the step of providing comprises providing the detailed source information.

A portion may be conceived as an excerpt from the source. As such, a portion may be a subset of the source. For instance, the portion may be parts of files, documents, or other contents of the source. The portion may comprise the data element and additional information or data—but in a more defined manner as in the source. In other words, the portion may be seen as being more specific than the source as it is focused on the (immediately) relevant context of data element. For instance, if the data element comprises one or more individual words or values, the source may comprise a specific text passage or snippet of the text document around the individual words or values which are relevant for interpreting the individual words/values.

The source provided by machine-learned models may be comparably coarse and comprise information which is irrelevant for the data element. Accordingly, it may still take considerable effort, e.g., by user, to retrace the data element in the source. By further pinpointing the data element in the source, this kind of information is minimized which may lead to a more efficient workflow and increase the data security.

According to some examples, the step of providing comprises providing the detailed source information to a user via a user interface. With that, the user is provided with more precise information as to where the data element can be found in the corpus of data.

According to an aspect, the verification function comprises a second machine-learned function different from the machine-learned function.

This may have the advantage that the verification function is able to detect and extrapolate patterns in the corpus of data or the source. Thereby, the second machine-learned function may comprise a different architecture than the machine-learned function and/or have undergone a different training than the machine-learned function, e.g., based on different training data. Therewith, the second machine-learned function may take a different “perspective” on the data element and the underlying data which can increase the reliability of the method.

According to some examples, the second machine-learned function comprises a natural language processing function. With that, second machine-learned function is better enabled to process unstructured natural language text in the input.

According to some examples, the second machine-learned function comprises a transformer network and/or a large language model (in particular, different than the transformer network or the large language model comprised in the machine-learned function).

According to some examples, the large language model of the second machine-learned function is an off-the-shelf large language model which is not further adapted to the task of providing a confidence measure. According to some examples, the large language model of the second machine-learned function may be selected from the models available at the date of filing such as GPT models (e.g., GPT-3.5 and GPT-4), PaLM, LLaMa, LLaMa 2, Falcon, Whisper, and the like.

According to some examples, the machine-learned function has been specifically adapted by training for identifying data elements, in particular, by a training process governed by the organization, e.g., within the premises of the organization, while the second machine-learned function has not been specifically adapted to the task of providing confidence measures, in particular, the second machine-learned function has not been further trained by the organization.

This has the advantage that the effort on the side of the organization applying the method can be reduced. At the same time, the independence of the second machine-learned function can be assured.

According to an example, the verification function may comprise a rule-based module configured to provide confidence measures. For instance, synonyms may be hard-coded in the rule-based module, in particular, based on one or more ontologies as herein described. This may have the advantage of a more rigorous quality control.

According to an aspect, the step of providing comprises: comparing the confidence measure to a predetermined criterion, if, based on the step of comparing, the confidence measure does not fulfill the predetermined criterion, the identified at least one data element to a user via a user interface (optionally: together with the confidence measure), receiving a user input directed to rejecting, accepting, or correcting the identified at least one data element, and providing the identified data element based on the user input.

The predetermined criterion may be conceived as a quality criterion for the data element which may be checked based on the confidence measure. Specifically, the predetermined criterion may be a metric, the confidence measure has to fulfill if the corresponding data element is to be regarded as matching a prompt or a data type. According to some examples, the criterion may be a threshold.

In other words, the above aspect provides a confirmation user interface dependent on the confidence measure. With that, a user may be specifically engaged for cases which are uncertain. In turn, the user does not need to be bothered for more secure cases. This increases the efficiency of the method without sacrificing the specificity.

According to some examples, the step of providing additionally comprises providing the source and/or the detailed source information.

The provision of the source and/or detailed source information helps the user to make an appropriate user input. Instead of having to review the corpus of data, the user may specifically focus on the source.

According to an aspect, the method further comprises further training the machine-learned function based on the user input. Further training may comprise adjusting the machine-learned function based on the user input. In the same way, also the verification function may be further trained based on the user input.

By adapting the machine-learned function and/or the verification function based on the user input, the algorithms involved can be continuously improved. This improves the accuracy and reliability of the method.

According to some examples, the method may comprise repeating the steps of applying the machine-learned function, of determining a confidence measure, and of providing the data element based on the user input, in particular, if the user input is directed to reject the data element.

According to some examples, the step of providing comprises comparing the confidence measure to a predetermined criterion, if, based on the step of comparing, the confidence measure does not fulfill the predetermined criterion, repeating the steps of applying the machine-learned function, of determining a confidence measure, and of providing the data element.

According to an aspect, the method further comprises receiving a natural language query from a user via a user interface, comparing the confidence measure to a predetermined criterion, if, based on the step of comparing, the confidence measure fulfills the predetermined criterion: generating a natural language answer to the query by applying a natural language generation function to the identified at least one data element, providing the answer to the user via the user interface.

The query may be a high level request or question from a user formulated in natural language. It may be input by a chat window or speech input module in the user interface. For instance, a query may look as follows “is patient A eligible for a treatment with medicament B”.

The natural language answer may be equally high-level and be output via the user interface, e.g., in a chat window. Staying in the above example an answer may be “no, patient A should not be treated with B because patient A is allergic to substance C”.

The natural language generation function may be identical to the machine-learned function. According to other examples, the natural language generation function may be different from the machine-learned function and/or the verification function. Thereby “different” may mean different in all aspects and examples as herein described in connection with the machine-learned function and the verification function. Specifically, the natural language generation function may be a large language model. More specifically, the natural language generation model may be an off-the-shelf large language model such as GPT models (e.g., GPT-3.5 and GPT-4), PaLM, LLaMa, LLaMa 2, Falcon, Whisper, and the like. According to some examples, the natural language generation function may be identical to the verification function.

Translating the data elements to answers provides the user with actionable results. Moreover, since the question answering is based on the quality-controlled retrieval of the data elements, the answers are highly reliable.

According to an aspect, the method further comprises receiving a natural language query from a user via a user interface, generating a natural language answer to the query by applying a natural language generation function to the identified at least one data element, providing the answer to the user via the user interface.

According to some examples, the step of accessing the corpus of data is based on the query. Specifically, the corpus of data may be accessed from a database based on the query. According to some examples, this may comprise extracting a data identifier from the query and querying the database on the basis of the data identifier.

For instance, according to the above example, a patient identifier may be extracted designating patient A in the database.

According to some examples, the step of obtaining the prompt is based on the query. This may comprise identifying data types required for answering the query. According to some examples, the data types may be identified based on a predetermined ontology of the kind as herein described. By obtaining the prompt based on the query, the prompt may be selectively adapted to the query which further improves the reliability of the result.

In the above example, a medical ontology may set out which allergies would rule out an administration of a certain medication. The corresponding data type may then be “allergy against substance C yes/no”. According to some examples, the data types may be identified by the machine-learned function (which is additionally configured for that purpose) or yet a further machine-learned function substantially as described in European application EP 23159107.4 the contents of which are incorporated herein in their entirety by reference.

According to some examples, the method further comprises selecting the data structure form a plurality of predetermined data structures based on the query. With that, an appropriate mapping of the corpus of data according to the query can be ensured.

According to some examples, the corpus of data relates to the electronic medical health record of a patient, and the data element relates to a medical finding.

An electronic medical health record may be the entirety of medical information electronically available for a patient in a certain organization. According to some examples, the medical health record may be stored in one or more databases within the organization. In particular, the one or more databases may comprise one or more local or distributed data storages. According to some examples, the one or more databases may comprise or may be embodied as a Picture Archiving or Communication System (PACS). Further, according to some examples, the one or more databases may comprise or may be embodied as an Electronic Heath Record (EHR) System. Further, according to some examples, the one or more databases may comprise or may be embodied as Radiology Information System (RIS) or Laboratory Information System (LIS). According to some examples, the one or more databases may be part of a healthcare information system operating in a given (healthcare) organization such a hospital or hospital chain.

With that, the method can be applied to the complex problem of data retrieval in the medical domain.

According to some examples, the corpus of data comprises at least one of a medical report with unstructured natural language text. According to some examples, the medical report may comprise at least one of a procedural report, a diagnostic report, and/or a lab report.

A procedural report may include a description of the procedures or treatments conducted or planned for a patient. A diagnostic report may include a description of medical findings of a patient and/or treatment recommendations. A lab report may comprise laboratory values of a patient such as blood values.

Medical reports comprise very relevant information for assessing a patient case. At the same time, they are often difficult to access by healthcare user due to their often unstructured nature. Here, due to the automated and quality-controlled retrieval of data elements, the method may effectively support.

According to some examples, the corpus of data comprises supplementary information of the patient.

According to some examples, the supplementary information may be information not comprised in the medical reports.

According to some examples, the supplementary information comprises one or more of the following elements:
medical image data such a whole slide images or radiology image data, and/or
a-priori knowledge of at least one type of cancerous disease, and/or
demographic data of the patient such as the patient's age or gender,
a lists of allergies, existing illnesses, comorbidities, risk factors and the like, and/or
a medical guideline applicable for the patient.

According to some examples, the method further comprises obtaining a context information for identifying the data element, wherein the machine-learned function is configured to identify the data element additionally based on the context information. According to some examples, the verification function may be configured to provide the confidence measure based on the context information.

According to some examples, the context information may be a diagnostic task to be performed for a patient, a procedure request for a patient, a clinical question to be answered for a patient, or a referral letter for a patient.

Based on the context information the machine-learned function can perform a more specific data retrieval which increases the reliability and efficiency of the method. Likewise, the verification function can work more reliably.

According to some examples, the source may be an excerpt of a medical report, such as a text passage or text snippet. The detailed source information may comprise one or more sentences of the text passage.

According to some examples, the question may be a medical question regarding the patient. Further, the answer may be a summary of the health state of the patient, in particular, in layman terms.

According to some examples the data structure is a data structure for providing a structured medical report. In other words, the method is enabled to automatically map unstructured medical data onto a structured medical report which increases the accessibility of the information available for a patient.

According to an aspect, the data structure is based on a medical ontology, in particular, SNOMED and/or RadLex, and/or the data structure is based on a communication standard in healthcare, in particular, FHIR and/or DICOM.

SNOMED (or SNOMED CT or SNOMED Clinical Terms) is a systematically organized computer-processable collection of medical terms providing codes, terms, synonyms and definitions used in clinical documentation and reporting. SNOMED is considered to be the most comprehensive, multilingual clinical health-care terminology in the world. Accordingly, the usage of SNOMED for the medical ontology facilitates an efficient and at the same time comprehensive data retrieval. For further information, reference is made to https://www.snomed.org/.

RadLex is a terminology with a focus on radiology the development of which is coordinated by the RSNA. Being specifically designed for radiology applications, the usage of RadLex facilitates an efficient and at the same time comprehensive data retrieval especially in PACS systems.

FHIR relates to the Fast Healthcare Interoperability Resources standard and is a set of rules and specifications for exchanging electronic health care data. It is designed to be flexible and adaptable, so that it can be used in a wide range of settings and with different health care information systems. The goal of FHIR is to enable the seamless and secure exchange of health care information, so that patients can receive the best possible care.

Whenever DICOM is mentioned herein, it shall be understood that this refers to the “Digital Imaging and Communications in Medicine” (DICOM) standard, for example according to the DICOM PS3.1 2020c standard (or any later or earlier version of said standard).

Basing the data structure on the above ontologies and/or standards ensures native support of the method for the prevailing standards in healthcare. This ensures the seamless integration of the processing results in clinical workflows.

According to an aspect, a method for providing a mapping of an unstructured corpus of data onto a predetermined data structure is provided. The method comprises a plurality of steps. One step is directed to provide a predetermined data structure with a plurality of different data types. Another step is directed to obtain the corpus of data. Another step is directed to provide a machine-learned function configured to map input data to data types. Another step is directed to apply the machine-learned function to the corpus of data so as to generate a mapping for one or more data elements in the corpus of data to one or more of the plurality of data types. Another step is directed to determine, for each mapping, a confidence measure using a verification function, the verification function being independent from the machine-learned function. Another step is directed to provide the mapping based on the confidence measure.

A mapping may be conceived as an assignment of a data element to a data type of the data structure. In other words, a data element may be identified as corresponding to (or being of) a data type of the data structure. Further, according to some examples, mapping may comprise populating the data fields of the data structure with the identified data elements.

The advantages of the above aspect correspond to the advantages described in connection with the respective features of the other examples or aspects as herein described. Further, the above aspect may be modified with the features of the other examples and aspects as herein described.

According to an aspect, a system for providing a data element is provided. The system comprises an interface unit and a computing unit. The computing unit is configured to obtain a prompt for providing the data element. Further, the computing unit is configured to access a corpus of data via the interface unit. Further, the computing unit is configured to host a machine-learned function configured to identify data elements in corpuses of data based on prompts. Further, the computing unit is configured to apply the machine-learned function to the corpus of data so as to identify at least one data element in the corpus of data corresponding to the prompt. The computing unit is configured to determine a confidence measure for the identified at least one data element using a verification function, the verification function being independent from the machine-learned function. The computing unit is configured to provide the identified at least one data element as the data element based on the confidence measure via the interface unit.

According to some examples, the computing unit, upon using the verification function, may be configured to call the verification function via the interface unit and/or transmit the data element and the underlying data (i.e., the corpus of data or the source) to the verification function via the interface unit. According to other examples, the computing unit, upon using the verification function may be configured to host the verification function.

The computing unit may be realized as a data processing system or as a part of a data processing system. Such a data processing system can, for example, comprise a cloud-computing system, a computer network, a computer, a tablet computer, a smartphone and/or the like. The computing unit can comprise hardware and/or software. The hardware can comprise, for example, one or more processors, one or more memories, and combinations thereof. The one or more memories may store instructions for carrying out the method steps according to the invention. The hardware can be configurable by the software and/or be operable by the software. Generally, all units, sub-units or modules may at least temporarily be in data exchange with each other, e.g., via a network connection or respective interfaces. Consequently, individual units may be located apart from each other. Further, the computing unit may be configured as an edge device. According to some examples, the computing unit may be localized within the premises of an organization in possession of (or administrating) the corpus of data.

The interface unit may comprise an interface for data exchange with one or more databases for retrieving a corpus of data from the one or more databases. The interface unit may be further adapted to interface with one or more users of the system, e.g., by displaying the result of the processing, e.g., any classification result, to the user (e.g., in a graphical user interface). The interface unit may be adapted to facilitate data communication within the premises of an organization. Further, the interface unit may be adapted to facilitate data communication to the outside of the organization, e.g., for calling the verification function, transmitting information to the verification function, and receiving the processing results of the verification function.

According to another aspect, the present invention is directed to a computer program product comprising program elements which induce a computing unit of a system configured for providing a data element to perform the steps according to one or more of the above method aspects and examples, when the program elements are loaded into a memory of the computing unit.

According to another aspect, the present invention is directed to a computer-readable medium on which program elements are stored that are readable and executable by a computing unit of a system configured providing a data element according to one or more method aspects and examples, when the program elements are executed by the computing unit.

The realization of the invention by a computer program product and/or a computer-readable medium has the advantage that already existing providing systems can be easily adapted by software updates in order to work as proposed by the invention.

The computer program product can be, for example, a computer program or comprise another element next to the computer program as such. This other element can be hardware, e.g., a memory device, on which the computer program is stored, a hardware key for using the computer program and the like, and/or software, e.g., a documentation or a software key for using the computer program. The computer program product may further comprise development material, a runtime system and/or databases or libraries. The computer program product may be distributed among several computer instances.

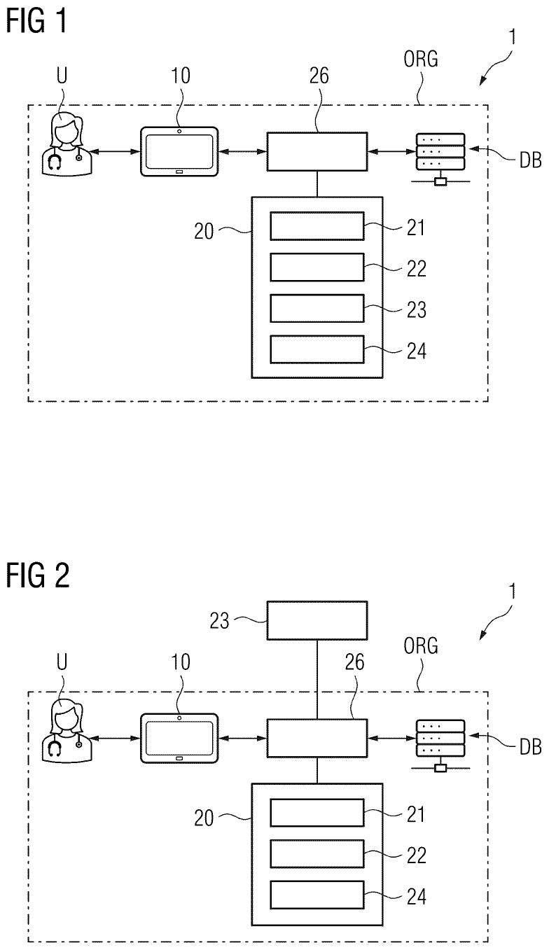

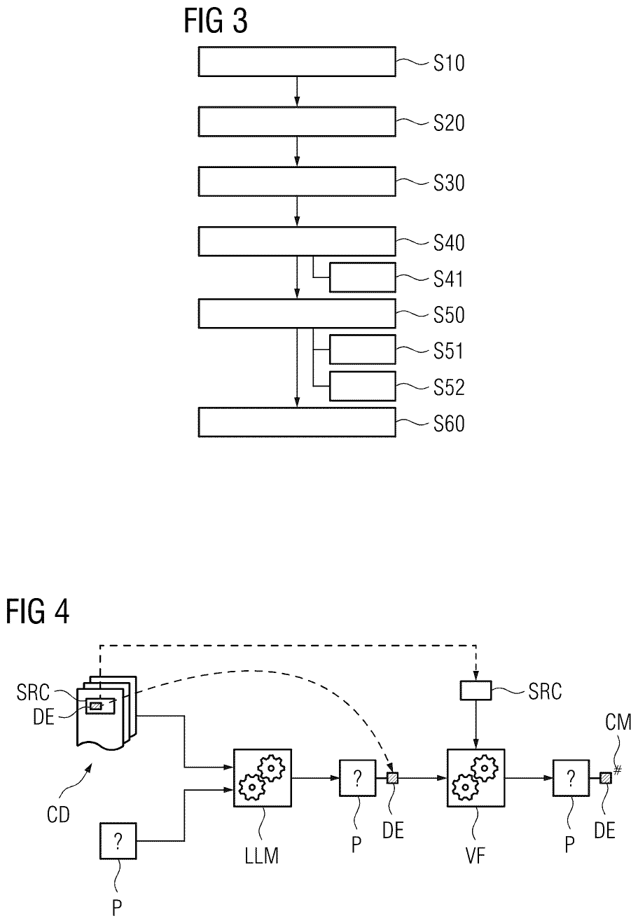

FIG. 1 depicts a system 1 for providing a data element DE and or a mapping MP of data elements DE to data types according to an embodiment. System 1 is adapted to perform the methods according to one or more embodiments, e.g., as further described with reference to FIGS. 3 to 11. A user of system 1, according to some examples, may generally relate to a healthcare professional such as a physician, clinician, technician, radiologist, pathologist and so forth.

System 1 comprises a user interface 10 (as part of the interface unit) and a computing unit 20. Further, system 1 may comprise or be connected to a database DB. Communication between the components may be provided by a data interface 26. The components 10, 20, DB may be part of a healthcare information system of an organization ORG. In the embodiment shown in FIG. 1, all components are within the premises of the organization ORG.

The database DB may generally be configured for acquiring and/or storing and/or forwarding medical data of one or more patients registered the system 1. The medical data may also be denoted as the electronic medical records of the respective patients. The database DB may be embodied by one or more storages. In particular, the database DB may be realized as a local or spread storage.

The electronic medical records may respectively comprise medical data of a patient. An electronic medical record may also be denoted as a corpus of data CD. For each patient registered in the system 1, there may be one electronic medical record or corpus of data CD in the database DB. The medical data stored in the database DB may comprise image and non-image data.

The image data may be three-dimensional image data sets acquired, for instance, using an X-ray system, a computed tomography system or a magnetic resonance imaging system or other systems. The image information may be encoded in a three-dimensional array of m times n times p voxels. Further, image data may comprise two-dimensional medical image data with the image information being encoded in an array of m times n pixels. According to some examples, these two-dimensional image data may have been extracted from three-dimensional medical image data sets. According to other examples, two-dimensional medical image data may have been generated by dedicated imaging modalities such as slide scanners used in digital pathology.

The non-image data may be procedural or diagnostic information providing additional information relating to the patient. The non-image data may relate to non-image examination results such as lab data, vital signs records (comprising, e.g., ECG data, blood pressure values, ventilation parameters, oxygen saturation levels) and so forth. Moreover, the non-image data may comprise structured and unstructured medical reports relating to prior examinations of the patient. Further, non-image data may comprise personal information of the patient such as gender, age, weight, insurance details, and so forth.

User interface 10 may comprise a display unit and an input unit. User interface 10 may be embodied by a mobile device such as a smartphone or tablet computer. Further, user interface 10 may be embodied as a workstation in the form of a desktop PC or laptop. The input unit may be integrated in the display unit, e.g., in the form of a touch screen. As an alternative or in addition to that, the input unit may comprise a keyboard, a mouse or a digital pen, a microphone, and any combination thereof.

User interface 10 may further comprise an interface computing unit configured to execute at least one software component for serving the display unit and the input unit in order to provide a graphical user interface GUI for allowing the user to review identified data elements DE or mappings MP and/or make user inputs U-INPT. In addition, the interface computing unit may be configured to communicate with the computing unit 20 for receiving the identified data elements DE or mappings MP and any relevant information for the identification/mapping process such as the source SRC or the detailed source information. The user may activate the software component via user interface 10 and may acquire the software component, e.g., by downloading it from the computing unit 20 or from an internet application store.

The interface computing unit may be a general processor, central processing unit, control processor, graphics processing unit, digital signal processor, three-dimensional rendering processor, image processor, application specific integrated circuit, field programmable gate array, digital circuit, analog circuit, combinations thereof, or other now known devices for processing image data. User interface 10 may also be embodied as a client.

Computing unit 20 may comprise sub-units 21-24 configured to process corpora of data CD in order to identify data elements DE according to one or more predefined data types in the corpora of data CD and, thus, provide a mapping MP of the respective corpora of data CD onto the predefined data types (which data types may be part of an overarching data structure DS).

Computing unit 20 may be a processor. The processor may be a general processor, central processing unit, control processor, graphics processing unit, digital signal processor, three-dimensional rendering processor, image processor, application specific integrated circuit, field programmable gate array, digital circuit, analog circuit, combinations thereof, or other now known device for processing image data. The processor may be single device or multiple devices operating in serial, parallel, or separately. The processor may be a main processor of a computer, such as a laptop or desktop computer, or may be a processor for handling some tasks in a larger system, such as in the medical information system or the server. The processor is configured by instructions, design, hardware, and/or software to perform the steps discussed herein. The computing unit 20 may be comprised in the user interface 10. Alternatively, computing unit 20 may be separate from user interface 10. Computing unit 20 may comprise a real or virtual group of computers like a so called ‘cluster’ or ‘cloud’. Such server system may be a central server, e.g., a cloud server, or a local server, e.g., located on a hospital or radiology site. Further, processing system 20 may comprise a memory such as a RAM for temporally loading data the database DB. According to some examples, such memory may as well be comprised in user interface 10.

Sub-unit 21 is a data retrieval module or unit. It is configured to access and search the database DB for the corpus of data CD (the medical record of a patient). Specifically, sub-unit 21 may be configured to formulate search queries and parse them to the database DB.

Sub-unit 22 may be conceived as a mapping module or unit. Sub-unit 22 is configured to identify data elements DE in corpora of data CD according to one or more data types. With that, sub-unit 22 maps the corpora of data CD to one or more data types. The assignment of data elements DE in a corpus of data to predetermined data types may be provided in the form of mappings MP and/or in the form of a data structure DS populated with the identified data elements DE. Further, sub-unit 22 may be configured to provide a source SRC in the corpus of data for each data element DE. Sub-unit 22 may be configured to host and run a machine-learned function LLM which has been adapted for identifying data elements DE in, in particular, unstructured, data according to predetermined data types.

Sub-unit 23 may be conceived as a verification module or unit configured to scrutinize that the mappings MP provided by sub-unit 22 are correct, or, in other words, that the data elements DE actually match the data types they have been assigned to. Sub-unit 23 is configured to quantify this assessment in a confidence measure CM. In order to provide this output, sub-unit 23 may be configured to process the source SCR as identified by sub-unit 22 and analyze the source SCR for an indication of the underlying data type. Sub-unit 23 may be configured to host and run an appropriately configured verification function VF which has been adapted according to intended functionality of sub-unit 23.

Sub-unit 24 may be conceived as a post-processing module or unit. In particular, sub-unit 24 may comprise a user interaction module or unit. Sub-unit 24 may be configured to bring data elements DE to the attention of a user U if an underlying mapping MP does not fulfill a certain quality criterion. Further, sub-unit 24 may be configured to collect a corresponding user input U-INPT from the user U which user input U-INPT may be directed to accept, reject, or correct the data element DE/mapping MP which was found not quite fulfilling the quality criterion.

Further, sub-unit 24 may be configured to take-in natural language queries Q from the user U and answer these queries Q based on (verified) data mappings MP in the form of natural language answers A. To this end, sub-unit 24 may be configured to host and run a natural language generation function LLM which has been configured for suchlike purposes.

The designation of the distinct sub-units 21-24 is to be construed by way of example and not as a limitation. Accordingly, sub-units 21-24 may be integrated to form one single unit (e.g., in the form of “the computing unit”) or can be embodied by computer code segments configured to execute the corresponding method steps running on a processor or the like of Computing unit 20. The same holds true with respect to the interface computing unit. Each sub-unit 21-24 and the interface computing unit may be individually connected to other sub-units and/or other components of the system 1 where data exchange is needed to perform the method steps.

Computing unit 20 and the interface computing unit together may constitute the computing unit of the system 1. Of note, the layout of this computing unit, i.e., the physical distribution of the interface computing unit and sub-units 21-24 is, in principle, arbitrary. For instance, sub-unit 24 (or individual elements of it or specific algorithm sequences) may likewise be localized in user interface 10. The same holds true for the other sub-units 21-23. Specifically, computing unit 20 may also be integrated in user interface 10. As already mentioned, computing unit 20 may alternatively be embodied as a server system, e.g., a local server, e.g., located on a hospital or radiology site within the organization ORG. According to some implementations, user interface 10 could be designated as a “frontend” or “client” facing the user, while computing unit 20 could then be conceived as a “backend” or server. The computational power of the system may be distributed between the server and the client (i.e., user interface 10). In a “thin client” system, the majority of the computational capabilities exists at the server. In a “thick client” system, more of the computational capabilities, and possibly data, exist on the client.

Individual components of system 1 may be at least temporarily connected to each other for data transfer and/or exchange. User interface 10 communicates with processing system 20 via (data) interface 26 to exchange, e.g., the corpus of data CD, data elements DE, sources SCR or any user input U-INPT made. Further, computing unit 20 may communicate with the database DB in order to retrieve data via the data interface 26. Data interface 26 may be realized as hardware- or software-interface, e.g., a PCI-bus, USB or fire-wire. Data transfer may be realized using a network connection. The network may be realized as local area network (LAN), e.g., an intranet or a wide area network (WAN). Network connection is preferably wireless, e.g., as wireless LAN (WLAN or Wi-Fi). Further, the network may comprise a combination of different network examples. Interface 26 together with the components for interfacing with the user U be regarded as constituting an interface unit of system 1.

FIG. 2 depicts a system 1 for providing a data element DE and or a mapping MP of data elements DE to data types according to an alternative embodiment. Like the system 1 of FIG. 1 the system 1 shown in FIG. 2 is adapted to perform the methods according to one or more embodiments, e.g., as further described with reference to FIGS. 3 to 11. Like reference numerals in FIG. 1 and FIG. 2 denote identical or analog components.

The embodiment shown in FIG. 2 differs from the embodiment of FIG. 1 in that the verification module 23 is outsourced from the organization ORG. That is, the verification function VF is hosted outside of the premises of the organization ORG. For instance, the verification module 23 may be implemented in the form of a web-service. Accordingly, this part of the functionality of system 1 may be a web application running in a web browser. Communication between the on-premises components of the organization ORG and sub-module 23 may be carried out using the https-protocol, for instance.

FIG. 3 depicts a method for providing a data element DE according to an embodiment. Corresponding data streams are illustrated in FIG. 4. The method comprises several steps. The order of the steps does not necessarily correspond to the numbering of the steps but may also vary between different embodiments of the present invention. Further, individual steps or a sequence of steps may be repeated.

At step S10, the prompt P is obtained. The prompt P is directed to find one or more data elements DE in the corpus of data CD. The one or more data elements DE relate to a certain kind of information which is sought for by a user U or for further processing. Obtaining the prompt P may mean evaluating the certain kind of information and determine which type(s) of data elements DE are needed to provide the certain kind of information. Accordingly, the prompt P may specify one or more data types for which corresponding data elements need to be identified.

The prompt P may be such that it can be processed by the machine-learned function LLM. The prompt P may be derived from the available information, such as any user input or context information. The context information may be an information about the patient such as a diagnostic task to be performed for the patient or a patient identifier.

At step S20, the corpus of data CD is accessed. This may comprise querying the databases 40 of the organization ORG for relevant files and/or retrieving those files. According to some examples, this may be based on a suitable data identifier such as patient identifier with which the patient is registered in the organization ORG.

At step S30, the machine-learned function LLM is provided. This may comprise holding the machine-learned function LLM function available in the processing system 20, for instance, as executable computer code.

At step S40, the machine-learned function LLM is applied to the corpus of data CD so as to identify one or more data elements DE according to the prompt P. In particular, the machine-learned function LLM may search for indications for the presence of certain data elements DE in the corpus of data CD.

At optional Step S41, the source SRC for the identified data elements DE in the corpus of data CD may be provided by the machine-learned function LLM. The source SRC is that portion of the corpus of data CD the machine-learned function LLM considers disclosing the identified data element DE. The source SCR may be a subset of the corpus of data CD such as an individual file or an excerpt from that file.

At step S50, the confidence measure CM is determined. The confidence measure CM may be seen as a measure for how well the data element DE satisfies the prompt P. In other words, the confidence measure CM may indicate if the identified data element DE corresponds to the data type asked for in the prompt P. The confidence measure CM may be a value or comprise a plurality of values in the form of a vector.

The confidence measure CM is determined by a verification function VF which is independent from the machine-learned function LLM. To derive the confidence measure CM, the verification function VF may be provided with the relevant information such as the identified data elements DE, the prompt P, and the corpus of data CD or parts thereof. This may mean that this information is transmitted to the verification function VF.

Specifically, the verification function VF may optionally be provided with the source SCR at optional step S51. With that, the verification function VF does not have to process the entire corpus of data CD but only parts thereof.

At optional step S52, the source SRC may further be used to derive a detailed source information which more precisely indicates where in the source SRC the data element DE comes from. The detailed source information may indicate a portion within the source SCR. The detailed source information may be output by the verification function VF as by-product of the confidence measure CM.

At step S60, the identified data element DE is provided. This may mean providing the data element DE to a user U via a user interface 10. The data element DE may be provided together with the corresponding confidence measure and the source SCR. Optionally, also the detailed source information may be provided in this context (optional step S65). Further providing may mean making the data element DE, the confidence measure CM, the source SCR, and/or the detailed source information available for further processing.

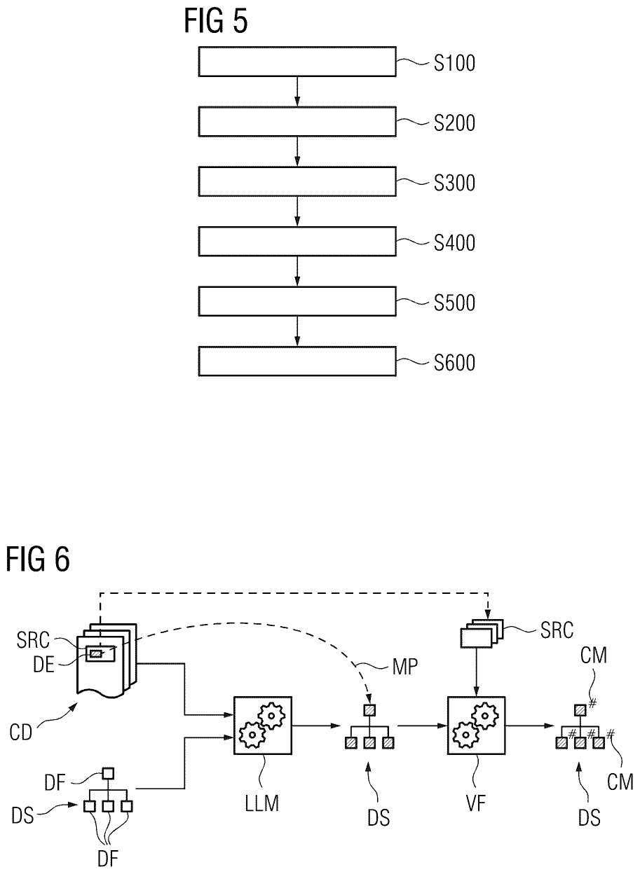

FIG. 5 depicts a method for providing a mapping MP of an unstructured corpus of data CD onto a predetermined data structure DS according to an embodiment. Corresponding data streams are illustrated in FIG. 6. The method comprises several steps. The order of the steps does not necessarily correspond to the numbering of the steps but may also vary between different embodiments of the present invention. Further, individual steps or a sequence of steps may be repeated.

The method according to FIGS. 5 and 6 follows the same concept as the method of FIGS. 3 and 4. However, the functionality is used for mapping unstructured data CD to predetermined data structure DS or for mapping data CD of a (or formatted according to) a first structure to a predetermined data structure DS different from the first data structure. As shown in FIG. 6, a data structure may have a plurality of data fields DF which may have a (logical) relation to one another. Specifically, the predetermined data structure DS may have a tree structure. Each data field DF may correspond to a data type. Accordingly, the data fields DF may be populated by data elements DE from the corpus of data CD. That way, the corpus of data CD may be mapped onto the data structure DS. This makes is possible to map unstructured data to a data structure DS or to map data formatted in one data structure to a different data structure DS.

At step S100, the data structure DS is provided. This may comprise selecting the data structure DS from a plurality of predetermined data structures DS which are, e.g., available in the organization ORG. The selection may be based on the context of the mapping task to be performed. In particular, the selection may be based on a query Q received from the user U or a diagnostic task to be performed.

At step S200, the corpus of data CD is obtained. This step may substantially correspond to step S20. At step S300, the machine-learned function LLM is provided. Also this step may substantially correspond to the analog step S30 of the method described in connection with FIGS. 3 and 4.

At step S400, the machine-learned function LLM is applied in substantially the same way as described in connection with step S40. That way, a mapping MP is generated linking data elements DE of the corpus of data CD to data types and therewith data fields DF of the data structure DS. Further, for each mapping MP, a source SRC may be provided substantially as described in connection with step S41.

At step S500, a confidence measure CM is provided for each mapping MP. In other words, a confidence measure CM is obtained for each data element DE. The confidence measures CM as such may be provided in substantially the same was as described in connection with step S50. Optionally, this may include providing the source(s) SCR of each mapping MP to the verification function VF (according to step S51) and, in turn, obtain a detailed source information for each mapping MP (according to step S52).

Finally, at step S600, the mappings MP are provided, optionally together with the confidence measures CM, the sources SCR, and/or the detailed source information for each mapping MP. As shown in FIG. 6, the mappings may be provided in the form of a populated data structure DS.

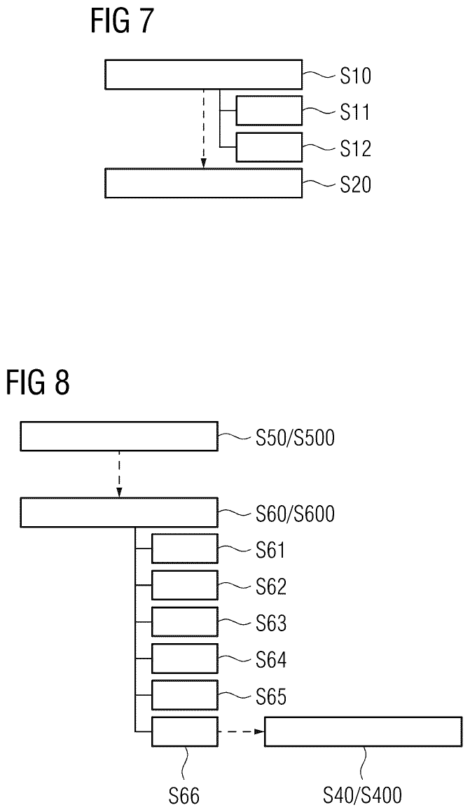

In FIG. 7, a modification of the method according to FIGS. 3 and 4 is shown. In the modification, the data structure DS is used to for defining the prompt P at step S10. The other steps correspond to the method shown in FIGS. 3 and 4.

Specifically, at step S11, a data structure DS is obtained. As described in connection with step S100, this may comprise selecting the data structure DS from a plurality of predetermined data structures DS which are, e.g., available in the organization ORG. The selection may be based on the context of the mapping task to be performed. In particular, the selection may be based on a query Q received from the user U or a diagnostic task to be performed.

That followed, at step S12, the prompt P may be defined based on the data structure DS. Specifically, the prompt P may defined as a prompt to retrieve data elements DE corresponding to a plurality of data types comprised in the data structure DS.

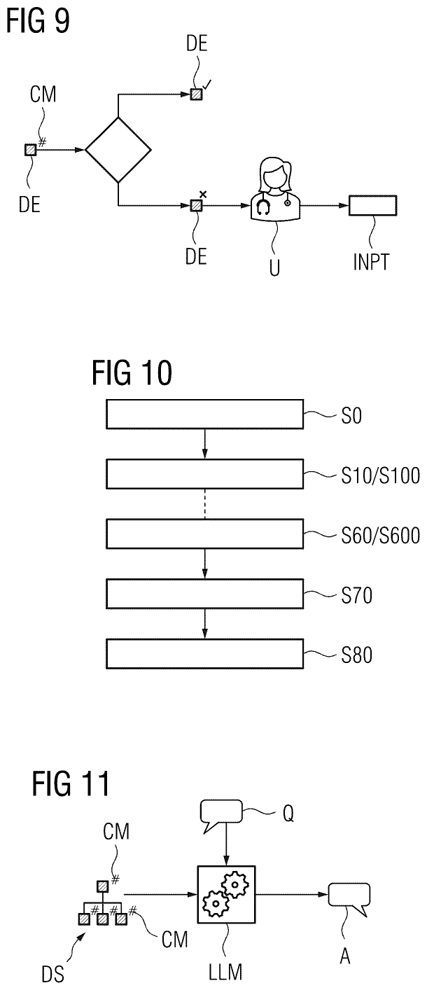

FIG. 8 depicts optional sub-steps directed to a further processing of the identified data elements DE and/or mappings MP. In particular, this may include user interaction workflows. Corresponding data streams are illustrated in FIG. 9. The steps may be individually or in arbitrary combinations executed within the steps S60 or S600.

At step S61, the confidence measure CM may be compared to a predetermined criterion. The predetermined criterion may be a quality metric. If the confidence measure CM fulfills the predetermined criterion, it can be concluded that the identified data elements DE correspond to the prompt P/the data types sought for.

If the confidence measure CM does not fulfill the predetermined criterion, the corresponding data element DE and relevant related information, i.e., the source SRC, the data type, and/or the detailed source information, are forwarded to the user U via user interface 10 at step S62.

At step S63, a user input U-INPT is received from the user U via user interface 10. The user input U-INPT may be directed to accept or reject the data element DE. Further, the user input U-INPT may be directed to correct the data element DE, e.g., by adapting or replacing its contents.

At step S64, the user input U-INPT may be processed. This may mean that the data element DE is provided based on the user input U-INPT. For instance, if the data element got rejected by the user U, it is not forwarded for further processing—in contrast to a case where it got accepted. If the user U corrected the data element DE, the corrected version is provided und used further.

At optional step S65, the source SCR and, optionally, the detailed source information are provided as already indicated in connection with steps S60 and S600. The source SCR and detailed source information may be provided to the user U via user interface 10.

Optionally, at step S66, the steps of applying the machine-learned function LLM (S60/S600), determining the confidence measure CM (S50/S500) may be repeated at least for those data elements DE or mappings MP which did not fulfill the predetermined criterion and/or which the user U rejected with user input U-INPT.

Further, at optional step S67, the user input U-INPT may be used for further training the machine-learned function LLM and/or the verification function VF.

FIG. 10 depicts optional method steps in a method for providing a mapping MP of an unstructured corpus of data CD onto a predetermined data structure DS or a method for providing a data element DE according to an embodiment. Corresponding data streams are illustrated in FIG. 11. The optional steps may, individually or in arbitrary combinations, precede or follow the processing according to the methods of FIG. 3 or FIG. 5 and any modification of these methods as herein described.

In the workflow of FIG. 10, the previous concepts directed to the identification of data elements DE or mappings MP are embedded in a broader context of a natural language question answering workflow.

At step S0, a natural language query Q is received from the user U via the user interface 10. For instance, the query Q may be input in a chat window in the user interface 10. The query Q may be a question concerning the corpus of data CD.

At step S70, a natural language answer A is generated based on the data elements DE or mappings MP identified by way of the workflows according to steps S10/S100 to S60/S600. Thereby, the steps directed to the identification of the data elements DE or mappings MP may take the query Q into account. For instance, the prompt P may be engineered based on the query Q or the data structure DS may be selected based on the query Q. According to some examples, this may comprise identifying data types based the query Q which would be needed for answering the query Q.

Alternatively, the steps directed to the identification of the data elements DE or mappings MP can also be executed independently of the query Q. In particular, these steps may already have been executed before the query Q is received so that the data elements DE or mappings MP are already available at the receipt of the query Q.

In any case, a natural language answer A is generated based on the data elements DE or mappings MP. In other words, the specific information therein comprised may be analyzed for its relevance for answering the query Q and the relevant parts may be cast into a natural language answer A for the user U. This may be done by any suitable natural language generation function. In the example of FIG. 11, this is provided for by an additional functionality of the machine-learned function LLM. However, the natural language generation function may also be a different/separate function, in particular, an independent large language model.

Optionally, any data elements DE or mappings MP may be subjected to a quality check before they are used for formulating the answer A. This is done in optional step S71. Specifically, only those data elements DE or mappings MP may be used the confidence measure CM of which fulfills a predetermined criterion as herein described.

Finally, at step S80, the answer A is provided to the user U via user interface 10.

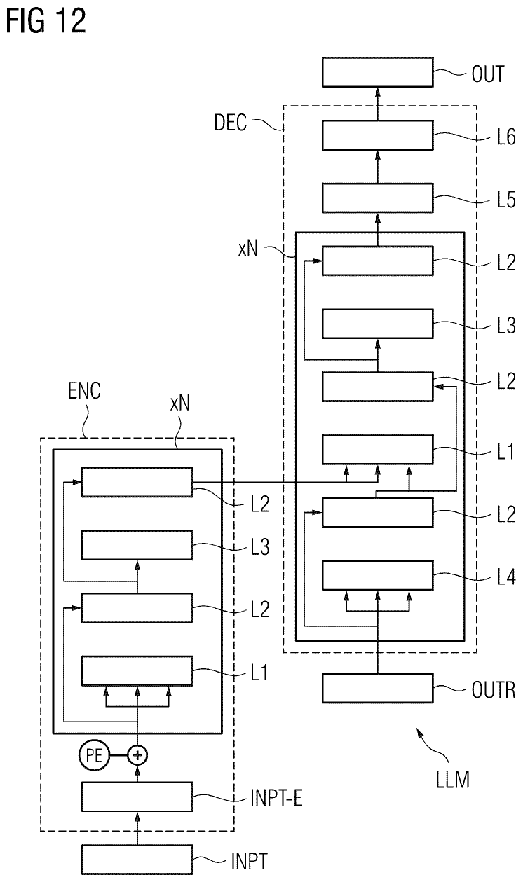

In FIG. 12, a schematic representation of the machine-learned function LLM according to an embodiment is shown. The machine-learned function LLM according to this embodiment comprises a transformer architecture. The transformer architecture follows an encoder-decoder structure and comprises an encoder ENC and a decoder DEC. In brief, the task of the encoder ENC is to map an input INPT to a sequence of continuous representations, which is then fed into a decoder DEC. The decoder DEC receives the output of the encoder ENC together with the decoder output OUTR at a previous iteration to generate an output OUT.

Specifically, the input INPT may be the corpus of data CD and an indication of the data types to identified in the corpus of data. The output OUT are the data elements DE which were identified according to the data types or, in other words, the mappings MP of data elements DE within the corpus of data CD to a predefined data structure DS comprising one or more data types.

The encoder ENC of this embodiment may comprise of a stack of N=6 identical layers. For the sake of easy reference, only one layer xN is shown in the drawing. Further, N may also be set to different values and, in particular, to values greater than N=6 according to the respective task. Each layer xN of the encoder ENC comprises two sublayers L1 and L3. The first sublayer L1 implements a so-called multi-head self-attention mechanism. Specifically, the first sublayer L1 may be configured to determine how relevant a particular word is with regard to other words in the input INPT. This may be represented as an attention vector. With that, it may be decided, if a certain passage in the corpus of data CD is related to a data type for which a data element DE is to be retrieved. To avoid any bias, multiple attention vectors per word may be generated and fed into a weighted average to compute the final attention vector of every word. The second sublayer L3 is a fully connected feed-forward network which may, for example, comprise two linear transformations with Rectified Linear Unit (ReLU) activation in between. The N=6 layers of the encoder ENC apply the same linear transformations to all the words in the input INPT, but each layer employs different weight and bias parameters to do so. Each sublayer L1, L3 is succeeded by a normalization layer L2, which normalizes the sum computed between the input fed into the respective sublayer L1, L3, and the output generated by the respective sublayer L1, L3 itself. In order to capture information about the relative positions of the words in the input INPT, positional encodings PE are generated based on input embeddings INPT-E prior to being fed into the layers xN. The positional encodings PE are of the same dimension as the input embeddings INPT-E and may be generated using sine and cosine functions of different frequencies. Then, the positional encodings PE may be simply summed to the input embeddings INPT-E in order to inject the positional information PE. Input embeddings INPT-E may be, as usual, a representation of each word in the input INPT, typically in the form of a real-valued vector that encodes the meaning of the word such that the words that are closer in the vector space are expected to be similar in meaning. According to some examples, a neural network may be used to generate the input embeddings INPT-E.

The output of the encoder ENC may be a numerical vector for each data item of the corpus of data CD indicating how relevant a particular data item of the corpus of data CD is with regard to a data type. The decoder DEC is configured to take this information and identify data elements DE. Put in highly simplified terms, the decoder DEC may use the most relevant parts as indicated by the encoder and derive a data element DE therefrom. At the same time, those most relevant part may be provided as source SRC.

Specifically, the decoder DEC of this embodiment may also comprise of a stack of N=6 identical layers xN each comprising three sublayers L4, L1, L3 which may be succeeded by a normalization layer L2 as explained in connection with the encoder ENC. For the sake of easy reference, only one layer xN of the decoder DEC is shown in the drawing. Further, N may also be set differently and, in particular, greater than N=6 according to the respective task. While the sublayers L1 and L3 of the decoder DEC correspond in their functionality to the respective sublayers L1 and L3 of the encoder ENC, sublayer L4 receives the previous output OUTR of the decoder DEC and implements multi-head self-attention over it weighing how important individual elements of the previous output vector OUTR are. That followed, the values from the first sublayer L4 of the decoder DEC are input in the L1-sublayer of the decoder DEC. This sublayer L1 of the decoder DEC implements a multi-head self-attention mechanism similar to the one implemented in the first sublayer L1 of the encoder ENC. On the decoder side, this multi-head mechanism receives the values from the previous decoder sublayer L4 and the output of the encoder ENC. Like in encoder ENC part, the output of the L1 sublayers is passed into a feed-forward layer L2, which will make the output vectors form into something which is easily acceptable by another decoder block or a linear layer. After all layers xN of the decoder DEC have been processed, the intermediate result is fed into a linear layer L5 which may be another feed-forward layer. It is used to expand the dimensions into a format expected for computing the output vector OUT, in this case a data element DE matching a data type and, optionally, a source SRC of the data element DE in the corpus of data CD. That followed, the result is passed through a Softmax Layer L6, which transforms the result into a data element DE of required format, e.g., in the form of a populated data structure DS.

Wherever meaningful, individual embodiments or their individual aspects and features can be combined or exchanged with one another without limiting or widening the scope of the present invention. In particular, while the systems and methods have been described in the description of embodiments with reference to medical use cases including the processing of medical data this is not to be construed as limiting the claims scope as the concepts, aspects, and examples are applicable to all kinds of data.

Advantages which are described with respect to one embodiment of the present invention are, wherever applicable, also advantageous to other embodiments of the present invention.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions on operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, similar or electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A computer-implemented method for providing a data element, the method comprising:
obtaining a prompt for providing the data element;
accessing a corpus of data;
providing a machine-learned function configured to identify data elements in corpora of data based on prompts;
applying the machine-learned function to the corpus of data to identify at least one data element in the corpus of data corresponding to the prompt;
determining a confidence measure for the identified at least one data element using a verification function, the verification function being independent from the machine-learned function; and
providing the identified at least one data element as the data element based on the confidence measure.

2. The method of claim 1, wherein:
the corpus of data comprises unstructured natural language text, and
the machine-learned function is configured to identify data elements in natural language text.

3. The method of claim 1, wherein the obtaining the prompt further comprises:
obtaining a predetermined data structure with a plurality of data types, and
defining the prompt as a prompt directed to identify data elements corresponding to one or more of the plurality of data types,
wherein the confidence measure is indicative of a correspondence between the identified at least one data element and the corresponding data type.

4. The method of claim 3, wherein
the data structure at least one of is based on an ontology of data types or comprises a tree structure of data types.

5. The method of claim 1, wherein
the machine-learned function is configured to provide a source in the corpus of data from which source the identified at least one data element was obtained,
the verification function is configured to provide confidence measures for identified data elements based on corresponding sources, and
the determining the confidence measure comprises inputting the source in the verification function.

6. The method of claim 1, wherein:
the machine-learned function is configured to provide a source in the corpus of data from which source the identified at least one data element was obtained,
the verification function is configured to derive detailed source information indicating portions within corresponding sources from which data elements have been obtained based on identified data elements and corresponding sources,
the determining the confidence measure comprises deriving a detailed source information indicating from which portion within the source the identified at least one data element has been obtained, and
the providing comprises providing the detailed source information.

7. The method of claim 1, wherein
the verification function comprises a second machine-learned function different from the machine-learned function.

8. The method of claim 1, wherein the providing comprises:
comparing the confidence measure to a predetermined criterion,
if the confidence measure does not fulfill the predetermined criterion
providing the identified at least one data element to a user via a user interface,
receiving a user input directed to rejecting,
accepting, or correcting the identified at least one data element, and
providing the identified data element based on the user input.

9. The method of claim 1, further comprising:
receiving a natural language query from a user via a user interface,
comparing the confidence measure to a predetermined criterion,
if the confidence measure fulfills the predetermined criterion
generating a natural language answer to the query by applying a natural language generation function to the identified at least one data element, and
providing the answer to the user via the user interface.

10. The method of claim 3, wherein:
the corpus of data relates to an electronic medical health record of a patient, and
the data element relates to a medical finding.

11. The method of claim 10, wherein at least one of
the data structure is based on a medical ontology, or
the data structure is based on a communication standard in healthcare.

12. A computer-implemented method for providing a mapping of an unstructured corpus of data onto a predetermined data structure, the method comprising:
providing the predetermined data structure with a plurality of different data types;
obtaining the corpus of data;
providing a machine-learned function configured to map input data to data types;
applying the machine-learned function to the corpus of data to generate a mapping for one or more data elements in the corpus of data to one or more of the plurality of data types;
determining a confidence measure using a verification function for each mapping, the verification function being independent from the machine-learned function; and
providing the mapping based on the confidence measure.

13. A system for providing a data element, the system comprising:
an interface unit; and
a computing unit, wherein the computing unit is configured to,
obtain a prompt for providing the data element,
access a corpus of data via the interface unit,
host a machine-learned function configured to identify data elements in corpora of data based on prompts,
apply the machine-learned function to the corpus of data to identify at least one data element in the corpus of data corresponding to the prompt,
determine a confidence measure for the identified at least one data element using a verification function, the verification function being independent from the machine-learned function, and
provide the identified at least one data element as the data element based on the confidence measure via the interface unit.

14. Computer program product comprising program elements that, when executed by a computing unit of a system, cause the system to perform the method of claim 1.

15. A non-transitory computer-readable medium comprising program elements that, when executed by a computing unit of a system, cause the system to perform the method of claim 1.

16. The method of claim 11, wherein
the data structure is at least one of SNOMED, RADLEX, FHIR or DICOM.

17. The method of claim 2, wherein
the machine-learned function is configured to provide a source in the corpus of data from which source the identified at least one data element was obtained,
the verification function is configured to provide confidence measures for identified data elements based on corresponding sources, and
the determining the confidence measure comprises inputting the source in the verification function.

18. The method of claim 17, wherein:
the machine-learned function is configured to provide a source in the corpus of data from which source the identified at least one data element was obtained,
the verification function is configured to derive detailed source information indicating portions within corresponding sources from which data elements have been obtained based on identified data elements and corresponding sources,
the determining the confidence measure comprises deriving a detailed source information indicating from which portion within the source the identified at least one data element has been obtained, and
the providing comprises providing the detailed source information.
